(19) United States
(12) Patent Application Publication
(10) Pub. No.: US 2017/0224859 A1
(43) Pub. Date: Aug. 10, 2017

(54) TRACEABILITY AND MONITORING OF A STERILISATION CASE AND THE CONTENT OF SAME

(71) Applicant: ANALYTIC-TRACABILITE HOSPITALIERE, Denain (FR)

(72) Inventors: Geoffrey BRONINX, Neuville-sur-Escaut (FR); Mathieu COTEROT, Bouvignies (FR)

(21) Appl. No.: 15/514,570

(22) PCT Filed: Nov. 13, 2015
(86) PCT No.: PCT/FR2015/053067

§ 371 Date: Mar. 27, 2017

(30) Foreign Application Priority Data
Nov. 13, 2014 (FR) 14 60963

(51) Int. Cl. A61L 2/28; A61B 50/34; A61B 90/96; A61B 50/33

(52) U.S. Cl. CPC A61L 2/28; A61B 50/33; A61B 50/34; A61B 90/96

(57) ABSTRACT

The present invention concerns, in this example, a sterilization case (200) for surgical instruments (210) including an electronic identity device (100) for the monitoring and traceability of the case and the content of same, the electronic device (100) including:—a measurement module (10) for taking at least one measurement relative to at least one parameter representative of an environmental condition to which the sterilization case (200) is exposed and for generating an item of digital measurement data (D_M) depending on the measurement,—a storage module (20) for storing the generated item of measurement data (D_M),—a communication module (30) suitable for cooperating with the storage module (20) in order to transmit the item of measurement data (D_M) to an external reader (300, 300′) when the external reader (300, 300′) interacts with the communication module (30).

### TECHNICAL FIELD AND PRIOR ART

The object of the present invention relates to the field of health safety, and more specifically to the traceability and monitoring of surgical instruments in a medical environment such as a hospital compound.

The goal of the object of the present invention is to meet the various health and safety standards that are currently in effect with regard to traceability and monitoring of surgical instruments in hospital compounds such as hospitals and clinics.

From the following description, it will be understood that the present invention has a particularly advantageous use in hospitals and clinics by allowing the traceability and the monitoring of surgical instruments for example all the way to the operating room.

Likewise, it will be understood that the present description has another advantageous use in veterinary clinics.

One of the advantageous uses of the present invention is that of precisely knowing the path of the surgical instruments during their cycle of use before their arrival in the operating room; in particular, one of the advantageous uses of the present invention is that of monitoring the phases of decontamination, disinfection and sterilisation of the surgical instruments before their arrival in the operating room; this is very important before a surgical procedure.

A surgical instrument in the context of the present invention should be understood here, throughout the following description, as any type of instrument or medical device intended to be used by a practitioner such as a doctor or surgeon during a medical or surgical procedure.

As non-limiting examples, this can be an instrument such as, in particular, an ancillary instrument, scalpel blade, forceps (hemostatic, cystoscopic, towel, intestinal, etc.), screw, ligature instrument, retractor or even probe; in this case, it is called an RMD, acronym for “Reusable Medical Device.”

It can also be an implant used during the procedure; then, it is called an IMD, acronym for “Implantable Medical Device.”

Today, hospitals and clinics are subject to very strict regulations and standards in terms of health safety.

Among the regulations and the standards currently in effect, Directive DGS/RI3/2011/449 from 1 Dec. 2011 recommends the individual traceability of reusable medical devices (or RMDs) in order to combat infections such as Creutzfeldt-Jakob disease and the variants thereof.

The goal of this directive is to follow up on the circular “138” from 2001, which requires the traceability of the last five patients for a surgical instrument.

Decree n° 2006-1497 specifies the scope of the traceability of medical devices by defining “the specific rules of materials vigilance carried out over certain devices.”

The text of this decree specifies that materials vigilance includes rules of traceability from the reception of the medical devices (MDs) in the hospital compound to their use on the patient.

The object of the present invention thus relates to the monitoring and traceability of the surgical instruments in hospital compounds, and in particular the monitoring and traceability of the surgical instruments from the reception thereof in the hospital compound to their use on the patient, in particular including their various phases of sterilisation (in washers, in autoclaves, etc.).

In other cases, the RMDs are temporarily loaned by the supplier laboratory. These RMDs are then returned after the surgical procedure.

In this case, it is understood that the object of the present invention also relates to the monitoring and traceability of these RMDs outside of the laboratory.

The subject matter of the present invention thus relates to containers, called sterilisation containers, for surgical instruments.

Such containers are routinely used by surgeons and/or associated medical assistance personnel to transport the medical instruments related to the procedure in question to the operating site, in general the operating room.

These sterilisation containers are arranged to hold the various surgical instruments required during the procedure.

These containers must also be made to be able to withstand, before and after use, the usual cleaning phases corresponding to a decontamination and sterilisation cycle, as is routine in the medical field, in particular before and after a surgical procedure.

In general, these decontamination and sterilisation phases are carried out in particular via an autoclave.

Here, conventionally, an autoclave is a container that closes hermetically and is configured to sterilise surgical instruments via steam (this is also called moist heat sterilisation).

Moist heat sterilisation must follow several specific phases (described in Regnault's table).

One of these phases involves raising the temperature to more than 134° C. and to more than 2.05 bar gauge (a little over 3 bar absolute). Another phase involves creating a vacuum.

The sterilisation containers are thus used both:

for storing and transporting the surgical instruments, and

for facilitating the phases of decontamination and sterilisation of these instruments before and after a surgical procedure.

Before the procedure and at the end of the procedure, the instruments are systematically cleaned, dried and then placed in sterilisation containers.

These containers are then inserted into a steriliser.

In general, the containers thus have perforations on at least one of their walls.

The steriliser generates a flow of vapour that penetrates each container by passing though these perforations.

The emission of this flow of vapour through the perforations allows the instruments contained in each container to be sterilised.

Such sterilisation containers are widely known in the prior art and are disclosed in particular in document FR 2 793 416, in document WO2005110268 and also in document U.S. Pat. No. 5,384,103.

The applicant has observed that there is a tendency towards the industrialisation of the entire process of retreatment of these sterilisation containers, and in particular the treatment relating to the phases of decontamination and sterilisation of the surgical instruments. Thus, specialised companies work as subcontractors for hospitals and clinics and deliver these sterilisation containers ready to use (that is to say, pre-arranged, decontaminated and sterilised).

It is therefore very important for hospitals and clinics to be able to carry out quality control on the sterilisation and the decontamination of these containers by service providers.

Moreover, it is noted that although the techniques for retreating these instruments have evolved and have been perfected with the appearance of automatic washer-disinfectors and high-performance sterilisers, the applicant maintains that these sterilisation containers as such have not really evolved.

The applicant has noted in particular that the documents mentioned above relate substantially to mechanical aspects with, for example, the arrangement of the perforations to make the containers modular.

Thus, despite all the regulations and standards currently in effect, it is very difficult at present to know the specific composition of a sterilisation container and the history of the instruments it contains; this is even more so when subcontracting companies are tasked with the decontamination and the sterilisation of the surgical instruments and deliver the pre-arranged, decontaminated and sterilised sterilisation containers for a pre-programmed surgical procedure.

Indeed, although the traceability computer system marketed by the applicant under the brand named ANCITRACK® and disclosed in document WO 2014/167252 allows the monitoring and traceability of the (unmarked) surgical instruments in a hospital compound to be managed, at present there is no integrated solution for the global management of the monitoring and traceability of a pre-arranged sterilisation container, in particular when the latter arrives in the hospital compound ready to use, that is to say, already decontaminated and sterilised for a surgical procedure.

### OBJECT AND SUMMARY OF THE PRESENT INVENTION

The present invention aims to improve the situation described above.

One of the goals of the present invention is to overcome the various disadvantages mentioned above by providing reliable technology for the monitoring and traceability of the surgical instruments in a hospital compound.

For this purpose, the object of the present invention relates, according to a first aspect, to a sterilisation container for surgical instruments. This container advantageously comprises an electronic identity device for the monitoring and traceability of the container and of the surgical instruments it contains.

According to the invention, the electronic device comprises:

a measurement module that is configured to take at least one measurement relating to at least one parameter representative of an ambient condition (temperature, pressure, humidity, shock, etc.) to which the sterilisation container and the surgical instruments are subjected and generate a piece of digital measurement data on the basis of the measurement taken,

a memorisation module that is configured to memorise the piece of measurement data generated,

a communication module suitable for cooperating with the memorisation module in order to transmit the piece of measurement data to an external reader when the external reader interacts with the communication module.

Preferably, the communication module is remote with respect to the measurement module in order to prevent interference.

Advantageously, the sterilisation container comprises a protective case enclosing said electronic identity device.

Preferably, said electronic identity device is at least partially formed from an impermeable and thermally insulating material in order to protect the electronic identity device, in particular when passing through an autoclave.

Thus, due to this arrangement of technical means, which is characteristic of the present invention, the container is an intelligent sterilisation container that can communicate with an external reader, for example an optical reader or an RFID or Bluetooth reader, in order to transmit, regardless of the ambient conditions, the information relative to the path and to the conditions to which the container has been subjected from the moment it was arranged to its arrival in the operating room.

The hospital staff can thus know, at any moment and in particular from the reception of the container up to the day of the surgical procedure, the state of the surgical instruments: are the instruments contained in the container still sterilised? What does the container contain? Have the instruments contained in the container potentially been contaminated?

The electronic identity device is protected from its outside environment and allows the information on the history of the sterilisation container to be transmitted.

The case that protects it allows it to resist the conditions to which it is subjected, for example when passing through an autoclave. To obtain the information relative to the history of the sterilisation container, it suffices for one of the members of the hospital staff to take a reader suitable for this purpose and interact with the communication module in order to obtain the desired relevant information.

The sterilisation container according to the invention therefore allows procedure control to be carried out by measuring at least one of the ambient parameters to which the container is subjected during its sterilisation path, and thus allows it to be determined whether the container has been correctly passed through the autoclave.

Passing through an autoclave is one of the steps of the sterilisation and decontamination phase that is verified. Other steps can also be verified (washing, rearranging, autoclaving, packaging, storage, etc.).

Advantageously, the protective case is at least partially formed from a material suitable for resisting pressures substantially between approximately 0 and 16 bar and/or temperatures substantially between approximately −20° C. and 200° C.

Preferably, the protective case comprises two half-shells rigidly assembled to each other with a sealing joint.

Preferably, these two half-shells are solution-dyed.

In a particular embodiment, the half-shells and the sealing joint are at least partially formed from at least one of the polymers chosen from the following: polysulfones, polyphenylsulfone resins, polyetherimides, polyamides, polyformaldehyde, butylene polyterephthalate.

The above polymers are particularly valued for their resistance to pressure, high temperatures and humidity.

Ceramic half-shells can also be provided.

The structure and the composition of this case allow good resistance to the conditions undergone when passing through an autoclave.

According to a particular embodiment, each of the two half-shells has an inner wall having a specific shape that acts as a radiator in order to cool the half-shells from the inside or slow down the absorption of outside heat by the case.

Preferably, the inner wall of the half-shell comprises an additional layer made from a thermally insulating material.

Such a material can, for example, be formed from silicic acid, epoxy resin or a ceramic material in order to capture the heat arriving from outside the case. Preferably, the measurement module is synchronised with an internal clock in such a way that the piece of digital measurement data is timestamped.

In other words, due to this synchronisation with an internal clock, the piece of digital measurement data comprises a piece of timestamp information relative to the date and time at which the measurement was taken.

It is thus possible to establish the history of the conditions to which the sterilisation container and the instruments it contains have been subjected.

It is also possible to know the exposure times, for example the time of exposure to a predetermined threshold temperature or pressure.

In one embodiment, the measurement module is configured to take measurements continuously.

This provides a complete report of the information relating to the conditions to which the container has been subjected.

In an alternative embodiment, the measurement module is configured to take measurements periodically according to a predetermined period.

This allows the number of measurements to be limited in order to limit the memory space required.

Preferably, the measurement module comprises a temperature probe.

This allows a piece of information relative to the temperatures to which the container and the instruments it contains have been subjected to be obtained. It is thus possible to verify, for example, that the temperature required for the sterilisation cycle to comply with the standards in effect has been respected.

In a particular embodiment, the temperature probe comprises a thermostat configured to detect an ambient temperature outside of said protective case greater than a predetermined threshold temperature between 100° and 130° Celsius.

Preferably, the protective case comprises an insert made from a material suitable for conducting heat, such as metal for example, and the thermostat is in direct contact with said insert.

Alternatively, the protective case has a thinner section of wall, and the thermostat is placed against the inner wall of the protective case at the thinner section of wall.

Preferably, the measurement module comprises a pressure sensor.

Likewise, this allows a piece of information relative to the pressures to which the container and the instruments it contains have been subjected to be obtained.

It is thus possible to verify, for example, that the pressure required for the sterilisation cycle to comply with the standards in effect has been respected.

Preferably, the measurement module comprises a humidity sensor.

Again likewise, this allows a piece of information relative to the humidity levels to which the container and the instruments it contains have been subjected to be obtained.

Preferably, the measurement module comprises a vibration sensor.

Here, this allows a piece of information to be obtained relative to the possible shocks to which the container and the instruments it contains have been subjected, for example during transportation within the hospital compound. It is thus possible to determine whether the surgical instruments, and in particular the implants, for example, have experienced a shock that could alter their mechanical performance.

Preferably, the measurement module comprises an accelerometer.

The use of an accelerometer allows:

the detection of possible shocks and thus possible deterioration of the sterile packaging of the case, and

the detection of when the sterilisation container is moving (handled by an operator) or stopped (in a standby or storage area).

It is of course understood that the measurement module can comprise all of the above sensors or only some of them.

The electronic identity device thus forms a sort of improved “black box” with sensors and a memorisation module containing all the information relative to the conditions to which the sterilisation container and the instruments it contains have been subjected.

It is thus possible, using the information recovered in this measurement module and stored in the memorisation module, to determine, for example, whether the decontamination and sterilisation cycles have indeed been respected, to determine whether the storage conditions (for example: the temperature and humidity conditions) have been respected, or to determine whether or not the instruments have been degraded during transportation (potential alterations in the instruments after an accidental fall, for example).

Optionally, the measurement module comprises sealing means.

In one alternative, a thermal insulation resin applied onto all or a portion of the measurement module is used. The purpose of this resin is to protect the components from heat.

It is understood here that such means allow the sealing of each of the sensors. With regard to the alternative in which the measurement module comprises a humidity sensor, it is understood here that the probe of this humidity sensor is in direct contact with the outside.

Advantageously, the communication module is suitable for communicating with a traceability computer system; such a system is suitable for identifying the various surgical instruments contained in a sterilisation container.

Such a computer system can be, for example, an ANCITRACK® system.

In this case, the communication module can receive, from this computer system, identity data containing the information relative to the various surgical instruments that the sterilisation container contains.

This identity data is memorised in the memorisation module.

The memorisation module thus contains all the information relative to the surgical instruments that the container contains and to the ambient conditions to which the container and these instruments have been subjected.

In an advantageous embodiment, the memorisation module comprises an electronic chip and the communication module comprises an antenna.

In this embodiment, the chip and the antenna together form a radio-frequency identity tag such as, for example, an RFID tag; thus, when a reader such as, for example, an RFID reader creates a magnetic or electric field near the antenna, the chip transmits the digital data that it contains to the reader via the antenna.

This embodiment is particularly advantageous for transmitting the relevant information to the medical staff provided with a suitable RFID reader.

Upon reception of the sterilisation containers, the medical staff merely has to read, via these radio-frequency means, the information contained in the chip in order to know the history of this container, in particular the history related to the conditions to which the container and the instruments it contains have been subjected.

Alternatively, the transmission of the information contained in the memorisation module to the reader can be carried out via a Bluetooth® connection.

In this case, the communication module comprises Bluetooth® or Bluetooth Low Energy wireless communication means. These communication means and the memorisation module cooperate in order to transmit the information to the reader.

In another embodiment that can be combined with one of the previous embodiments, the sterilisation container comprises, on one of its lateral walls, a reading zone having a matrix barcode.

In this embodiment, the optical reading of this code by an optical reader allows the transmission of the piece of digital measurement data to the reader.

Preferably, the communication module is suitable for communicating with the external optical reader via a wireless communication protocol such as Bluetooth.

This protocol allows the sterilisation container to communicate directly with a large number of professional and consumer devices.

It also allows greater distances to be covered than with conventional RFID.

Moreover, it is considered to be better able to pass through metal than RFID.

Finally, it is noted that the latest versions have energy-saving modes highly suitable for use, in particular when the sterilisation container is stored for a prolonged period (several months/years).

Preferably, the container comprises on-board intelligence.

Thus, a processing module is provided (for example a processor or a microprocessor or a microcontroller, etc.) that is configured to process the digital measurement data memorised in the memorisation module.

This processing allows the real-time generation of a warning signal when the data contains a piece of information that does not comply with a predetermined vigilance rule.

Thus, it is possible to verify that the sterilisation phase has been carried out in compliance with the standards in effect by verifying that the container has indeed been subjected to a temperature greater than or equal to 134° C. for a predetermined time (for example 18 minutes).

This on-board intelligence allows the container to be autonomous; it is no longer necessary to use a reader to know whether the case can be used without the risk of contamination.

Advantageously, the electronic identification device comprises a warning module that cooperates with the processing module. This module is thus configured to emit a warning such as, for example, a light alert upon reception of a warning signal.

Advantageously, the electronic identification device comprises a rapid-recharge power supply module.

Preferably, the power supply module comprises a thermoelectric generator configured to convert the thermal energy provided when passing through an autoclave into electric energy.

Such a generator allows recharging when passing through an autoclave.

The power supply module can also be of the capacitor or super-capacitor type. In this case, there needs to be a base or a suitable recharging connection.

These various types of power supplies ensure the energy autonomy of the device. Alternatively, the power supply module is high-capacity, such as a battery, or autonomous, such as a solar module.

In correlation, the object of the present invention relates, according to a second aspect, to an assembly for the monitoring and traceability of surgical instruments; this assembly consists of the following elements:

a sterilisation container such as that described above comprising the instruments in question, and

an external reader suitable for communicating with the electronic identity device of said container.

Thus, the present invention, via its various structural and functional technical features, provides hospital bodies with intelligent, communicating sterilisation containers capable of recording the relevant information relating to the treatment to which these containers have been subjected, in particular during the decontamination and sterilisation phases.

### BRIEF DESCRIPTION OF THE APPENDED FIGURES

Other features and advantages of the present invention will be clear from the following description, with reference to the appended FIGS. 1 and 2 that illustrate an example of an embodiment of the present invention that is in no way limiting and in which:

FIG. 1 shows a perspective view of a sterilisation container according to an example of an embodiment of the present invention;

FIG. 2 shows a schematic view of a sterilisation container according to FIG. 1.

### DETAILED DESCRIPTION OF AN ADVANTAGEOUS EXAMPLE OF AN EMBODIMENT

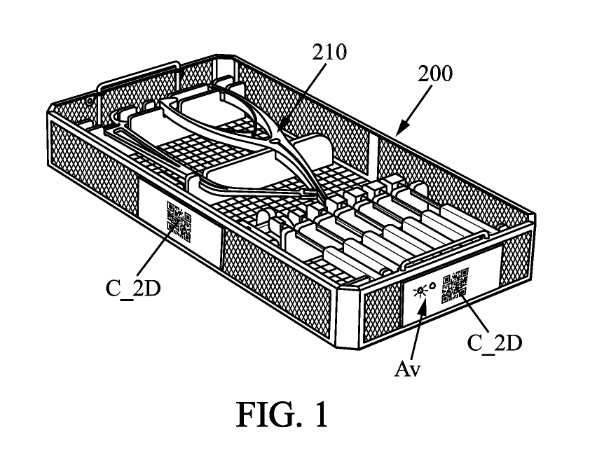

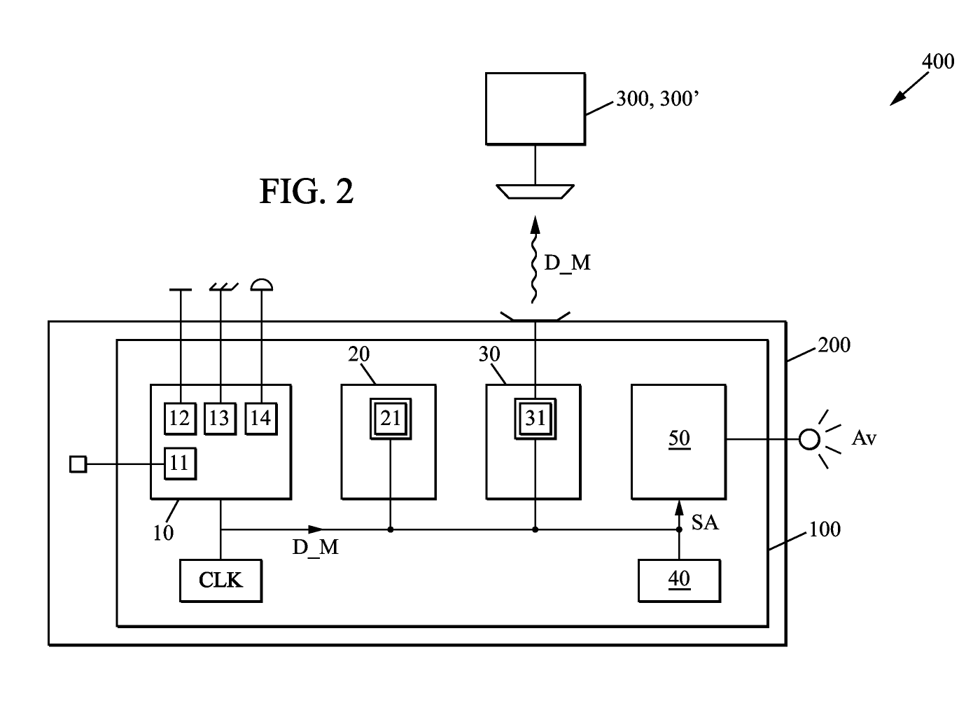

A sterilisation container according to an advantageous example of an embodiment will now be described below, with reference to both FIGS. 1 and 2.

Designing an intelligent and autonomous sterilisation container that allows the composition of said container and its history to be known at any time is one of the goals of the present invention.

The applicant has observed that in hospitals and in particular in the sterilisation department, the operations for accurately knowing the composition of a container are very complex.

Moreover, it is very difficult to know and accurately verify the state of a container and of the instruments that it contains after prolonged storage; here, state means to verify that the container and the instruments that it contains have been stored and preserved in conditions that prevent any potential contamination.

Likewise, it is very difficult to verify that the decontamination and sterilisation cycles have been correctly carried out.

The underlying concept on which the present invention is based is that of designing a sterilisation container equipped with electronic and computer means acting as a “black box”.

This “black box” is an improved black box that must allow the relevant information (composition of the container, temperatures, pressures, humidity levels, shocks experienced, etc.) on the container 200 and on the instruments 210 that it contains to be provided at any time.

In the example described here, the first phase of disinfecting the surgical instruments 210 takes place in the operating room as soon as the operation is over or in a medical care department as soon as the procedure is over.

The instruments 210 used, which are contaminated, are submerged in a detergent-disinfectant solution.

This first precaution allows the proliferation of germs, the development of which is often exponential, to be combatted early on.

The instruments 210 are then handled:

either by the sterilisation department of the hospital;

or by a subcontracting service-provider company. In the example described here, the instruments 210 are then placed in a suitable piece of medical furniture (cabinets or carts) and pass through a professional medical washing machine such as a large washer/disinfector.

In the example described here, this machine operates with water called “reverse osmosis” water (that is to say, water from which scale and limestone have been chemically removed).

In the example described here, the protocol further comprises chemical washing at approximately 60° C. then disinfection at high temperature (approximately 93° C.).

These conditions must be meticulously respected in order to be compliant with health standards and prevent any health risk.

Upon exiting this machine, the instruments 210 have been decontaminated; they are then handled with great care under very strict hygiene conditions: the quality of the air is permanently controlled and employees wear special clothing in order to be able to carry out the step of reconditioning the instruments.

The operator in charge of the sterilisation of the instruments 210 then identifies each of the decontaminated instruments 210, for example using an ANCITRACK® apparatus or a WHITEREADER® apparatus.

This operator then places the instruments 210 in a sterilisation container 200 on suitable supports in order to form the container 200 for a programmed procedure.

This identification and rearrangement step allows a plan of the sterilisation container 200 to be established, with all the instruments 210 that it contains.

The computer system allowing this identification and rearrangement, for example ANCITRACK®, generates identity data containing the identification information that allows the monitoring and traceability of the container 200 and of the instruments 210 that it contains.

In the example described here, the complete container 200 thus rearranged is then:

either packaged in such a way as to respect the standards in effect, in sterile fields specifically designed for sterilisation (packaging that is impermeable once dry);

or inserted into a sealed container specially designed for sterilisation. Starting with this step, the approved container of these instruments is no longer visible and cannot therefore be controlled by conventional means (visual identification, WHITEREADER® or ANCITRACK®). In the example described here, the container 200 is then inserted into a steam autoclave; this sterilisation step involves bringing the container 200 to a temperature of 134° C. for a predetermined time, for example 18 minutes.

The pressure and humidity conditions are also controlled here in order to have sterilisation that meets the health standards in effect.

Once sterilised, the instruments 210 are ready to be used; the instruments 210 are then sent directly back to the users or are stored in suitable areas for an imminent upcoming operation.

These storage areas comply with very strict standards, in particular in terms of temperatures and humidity.

As indicated above, it is very difficult or even impossible to know the precise composition of the container for each container 200, or to know whether or not the decontamination, sterilisation or storage conditions have been respected or whether or not the integrity of the instruments 210 is still intact because of the packaging or the sealed container.

It is noted here that the field used is permeable to vapour once wet and becomes impermeable again once dry, the sealed containers are therefore containers having an aluminium filter (filter made from the same material as the packaging), and the filter is permeable to vapour once wet and becomes impermeable again once dry: for this reason, it is important to not exceed a certain humidity level during storage (doubts about the integrity of the impermeability).

Has the temperature of 134° C. for 18 minutes been reached during the sterilisation cycle? Has the humidity level during storage been respected? Today, it is impossible to answer these questions; this is even more so when these treatment services are subcontracted out to outside companies.

In the example described here, in order to overcome these various disadvantages, the container 200 is intended to be equipped with an electronic identity device 100.

This device 100 is characteristic of the present invention; it forms a real “black box” of the surgical instruments 210.

In the example described here, this device 100 consists of a measurement module 10. This module 10 is configured to continuously (or alternatively periodically) take at least one measurement relative to at least one parameter representative of an ambient condition to which the container 200 is subjected. In the example described here, these parameters are the temperature, the pressure, the humidity level, and the possible shocks to which the instruments have been subjected and that are capable of degrading the instruments.

It is understood here that other parameters can be probed in the context of the present invention.

In the example here, the measurement module 10 thus comprises a temperature probe 11, a pressure sensor 12, a humidity sensor 13, a vibration sensor 14 and an accelerometer (not shown here).

It is thus possible to know the temperature, pressure and humidity conditions, as well as the shocks to which the instruments 210 have been subjected, for example during the decontamination, cleaning or sterilisation cycles or during the phases of storage and/or transportation of the container 200.

These sensors 11, 12, 13 and 1 are configured to generate a piece of digital measurement data D_M on the basis of the measurement taken.

The digital data D_M is preferably timestamped.

This is made possible in the context of the present invention by a clock CLK internal to the device 100.

In the example described here, each of these sensors 11, 12, 13 and 14 is indeed synchronised with this clock CLK in order to have a timestamped piece of data D_M comprising, in particular, the time and date of the measurement.

This data D_M is then memorised by the memorisation module 20.

In the example described here, this module 20 comprises ROM electronic memorisation means.

These means thus store the measurement data D_M.

In the example described here, the device 100 provided in the container 200 further comprises a communication module 30.

In the example described here, this communication module 30 is suitable for communicating with the ANCITRACK® computer system in order to receive the identity data containing the information relative to the various surgical instruments 210 that the sterilisation container 200 contains.

This identity data is memorised in the memorisation module 20.

The memorisation module thus contains all of the information relative to the surgical instruments that the container contains and to the ambient conditions to which the container and these instruments have been subjected. This module 30 is preferably suitable for cooperating with the memorisation module 20 in order to transmit all of the measurement data D_M to an external reader 300 or 300′ when the external reader 300 or 300′ interacts with the communication module 30.

In the example described here, this module 30 comprises an antenna 31.

In this example, the memory module 20 may also comprise an electronic chip 21.

The chip 21 and the antenna 31 thus form an RFID radio-identification tag.

Thus, in order to know the information contained in the memorisation module 20 (here, the chip 21), the operator merely has to take an RFID reader 300 and recover the data D_M by bring this reader 300 close to the antenna 31.

The reader 300 generates an electromagnetic (or electric) field that then allows the transmission of the data D_M contained in the chip 21.

In other words, in this example, the communication module 30 comprises a receiver 31 of wireless communication such as an RFID antenna.

Other transmission modes can be imagined, for example BLE wireless communication (for “Bluetooth Low Energy”).

In this example, when a BLE reader 300 establishes communication with the receiver 31, the latter then transmits the digital data D_M contained in the memorisation module 21 to the reader.

In the example described here, it is also possible for the reading to be carried out via optical reading of a “DataMatrix” two-dimensional matrix barcode C_2D; such a code C_2D is present on at least one reading zone of the container 200.

In this mode, the operator scans the code C_2D and thus recovers all of the data D_M stored in the module 20.

As mentioned above, the communication module 30 can also communicate with the ANCITRACK® system.

It is thus possible to recover the information relative to the plan of the instruments 210 in the container 200.

In the example described here, the electronic identity device 100 comprises on-board intelligence in order to be autonomous and be able to provide a first level of diagnostics with regard to the risks encountered.

The device 200 thus comprises a microprocessor 40 allowing the memorised digital measurement data D_M to be processed. This microprocessor 40 verifies that the data D_M does not contain a piece of information that would not comply with a predetermined vigilance rule, for example a temperature not reached during the sterilisation phase, an insufficient sterilisation time, a shock capable of degrading the instruments contained in the containers, etc.

These rules predefined by the standards in effect are stored for example in the memorisation module 20.

If one of the pieces of information contained in the data D_M does not comply with one of the predetermined vigilance rules, the microprocessor 40 generates a warning signal SA.

In the example described here, the warning module 50 of the device 100 emits an alert Av such as, for example, a light alert upon reception of such a warning signal SA.

Via this microprocessor 40 and this warning module 50, the operator that receives the container 200 can immediately see whether the container 200 has been subjected to outside conditions (temperature, pressure, etc.) that would not comply with predetermined rules.

In the example described here, the warning module 50 may comprise a set of diodes such as a “green” diode and a “red” diode, the green colour corresponding to a compliant state and the red colour corresponding to a non-compliant state.

Thus, via this device 100 consisting of these various electronic modules, it is possible to equip the container 200 with on-board intelligence that facilitates the work of the operators tasked in the hospital compound with monitoring the compliance of the instruments 210 before a surgical procedure.

To withstand the ambient conditions (temperature, pressure, humidity), in particular when passing through an autoclave, the device 100 is placed in a protective case 110 consisting of two rigid half-shells (not shown here).

Preferably, half-shells made from a polymer suitable for resisting high temperatures and high pressures are provided. It is also intended for these half-shells to be assembled together via a sealing joint.

It should be noted that this detailed description relates to a particular example of an embodiment of the present invention, but that in no case should this description be in any way limiting to the object of the invention; on the contrary, its goal is to eliminate any possible imprecision or any incorrect interpretation of the following claims.

1-20. (canceled)

21. A sterilization container for surgical instruments comprising an electronic identity device for the monitoring and traceability of said container and of the instruments that it contains, said electronic device comprising:
a measurement module configured to take at least one measurement relating to at least one parameter representative of an ambient condition to which said sterilization container and said instruments are subjected and generate a piece of digital measurement data on the basis of said measurement taken,
a memorisation module configured to memorise said piece of measurement data generated,
a communication module suitable for cooperating with said memorisation module in order to transmit said piece of measurement data to an external reader when said external reader interacts with said communication module,
wherein said sterilization container comprises a protective case enclosing said electronic identity device, said case being at least partially formed from an impermeable and thermally insulating material in order to protect said electronic identity device, in particular when passing through an autoclave.

22. The sterilization container according to claim 21, wherein the protective case is at least partially formed from a material suitable for resisting pressures substantially between approximately 0 and 16 bar and/or temperatures substantially between approximately −20° C. and 200° C.

23. The sterilization container according to claim 21, wherein the protective case comprises two half-shells rigidly assembled to each other with a sealing joint.

24. The sterilization container according to claim 23, wherein the half-shells and the sealing joint are at least partially formed from at least one of the polymers chosen from the following: polysulfones, polyphenylsulfone resins, polyetherimides, polyamides, polyformaldehyde, butylene polyterephthalate.

25. The sterilization container according to claim 21, wherein said measurement module comprises a temperature probe.

26. The sterilization container according to claim 25, wherein said temperature probe comprises a thermostat configured to detect an ambient temperature outside of said protective case greater than a predetermined threshold temperature between 100° and 130° Celsius.

27. The sterilization container according to claim 26, wherein said protective case comprises an insert passing all the way through, made from a material suitable for conducting heat, and wherein said thermostat is in direct contact with said insert.

28. The sterilization container according to claim 26, wherein said protective case has a thinner section of wall, and wherein said thermostat is placed against the inner wall of said protective case at said thinner section of wall.

29. The sterilization container according to claim 21, wherein said measurement module comprises a pressure sensor.

30. The sterilization container according to claim 21, wherein said measurement module comprises a humidity sensor.

31. The sterilization container according to claim 21, wherein said measurement module comprises a vibration sensor.

32. The sterilization container according to claim 21, wherein said measurement module comprises an accelerometer.

33. The sterilization container according to claim 21, wherein said measurement module is synchronised with an internal clock in such a way that said piece of digital measurement data is timestamped.

34. The sterilization container according to claim 21, comprising, on one of its lateral walls, a reading zone having a matrix barcode, the optical reading of said code by an external optical reader triggering the transmission of the piece of digital measurement data to said reader.

35. The sterilization container according to claim 21, wherein said communication module is suitable for communicating with said external optical reader via a Bluetooth wireless communication protocol.

36. The sterilization container according to claim 21, wherein the electronic identity device comprises a processing module configured to process the digital measurement data memorised in the memorisation module and generate a warning signal when the data contains a piece of information that does not comply with a predetermined vigilance rule.

37. The sterilization container according to claim 36, wherein the electronic identity device comprises a warning module that cooperates with the processing module and is configured to emit an alert upon reception of a warning signal.

38. The sterilization container according to claim 21, wherein said electronic device comprises a rapid-recharge power supply module.

39. The sterilization container according to claim 38, wherein said power supply module comprises a thermoelectric generator configured to convert the thermal energy provided when passing through an autoclave into electric energy.

40. An Assembly for the monitoring and traceability of surgical instruments, comprising:
a sterilization container according to claim 21 comprising said surgical instruments, and
an eternal reader suitable for communicating with the electronic identity device of said container.
